(19) United States
(12) Patent Application Publication
(10) Pub. No.: US 2025/0099224 A1
(43) Pub. Date: Mar. 27, 2025

(54) BIOLOGIC TUBE WITH WEAVED SUTURE ENHANCING LIGAMENT OR TENDON REPAIR

(71) Applicant: Patrick A. MASSEY, Shreveport, LA (US)

(72) Inventor: Patrick A. MASSEY, Shreveport, LA (US)

(21) Appl. No.: 18/783,657

(22) Filed: Jul. 25, 2024

Related U.S. Application Data
(60) Provisional application No. 63/540,238, filed on Sep. 25, 2023.

(51) Int. Cl. A61F 2/08; A61L 27/38

(52) U.S. Cl. CPC A61F 2/0811; A61L 27/3834; A61F 2002/0829; A61F 2002/0852; A61F 2210/0004; A61F 2220/0075

(57) ABSTRACT

A medical device includes a scaffold having at least two sides, a washer; and at least one suture having a medial portion passed through the washer, and two portions extending from the medial portion each weaved to a respective one of the two sides of the scaffold. The scaffold may include collagen and/or dermal allograft tissue. The scaffold may be shaped as a tube. Bone marrow aspirate may be embedded into an interior side of the tube. The two portions of the suture extending from the medial portion may be weaved to respective two diametrically opposed sides of the tube. A ring may be inserted into the tube at a tissue attachment side of the tube to keep the tube open. The ring may be formed of a bioabsorbable material. The scaffold may be a rectangular sheet.

### CROSS-REFERENCE TO RELATED APPLICATION

This application claims the benefit of priority of U.S. provisional patent application No. 63/540,238, titled “A BIOLOGIC TUBE REINFORCED WITH WEAVED SUTURE FOR ENHANCING LIGAMENT OR TENDON REPAIR WITH BIOLOGICS AND PROTECTING REPAIRS,” filed on Sep. 25, 2023, which is incorporated herein in its entirety by this reference.

### TECHNICAL FIELD

The present disclosure relates to devices and methods for repairing tendons and other biologic structures. More particularly, the present disclosure relates to a biologic tube reinforced with weaved suture for enhancing ligament or tendon repair with biologics and protecting repairs.

### BACKGROUND

Anterior cruciate ligament repairs and other ligament repairs need reinforcement as well as biological stimulation to heal. Without reinforcement, repairs are prone to failure. Current repair techniques have a high failure rate.

Current repair techniques do not provide any reinforcement to the repair. Additionally, they do not provide a conduit to protect clot formation and healing at the repair site of the ligament to bone.

### SUMMARY

This summary is provided to briefly introduce concepts that are further described in the following detailed descriptions. This summary is not intended to identify key features or essential features of the claimed subject matter, nor is it to be construed as limiting the scope of the claimed subject matter.

According to at least one embodiment, a medical device includes a scaffold having at least two sides, a washer; and at least one suture having a medial portion passed through the washer, and two portions extending from the medial portion each weaved to a respective one of the two sides of the scaffold.

The scaffold may include at least one of collagen and dermal allograft tissue.

The scaffold may be shaped as a tube.

Bone marrow aspirate may be embedded into an interior side of the tube.

The two portions of the suture extending from the medial portion may be weaved to respective two diametrically opposed sides of the tube.

A ring may be inserted into the tube at a tissue attachment side of the tube to keep the tube open.

The ring may be formed of a bioabsorbable material.

The scaffold may be a rectangular sheet.

The scaffold may include bone marrow aspirate.

The washer may have a hole on an end thereof for passing the at least one suture and

two slits in a middle thereof.

In at least one embodiment, a medical procedure includes repairing an anterior cruciate ligament using the medical device.

The medical procedure may include placing the tube around an anterior cruciate ligament.

The medical procedure may include placing the rectangular sheet next to an anterior cruciate ligament.

The above summary is to be understood as cumulative and inclusive. The above and below described features are to be understood as combined in whole or in part in various embodiments whether expressly described herein or implied by at least this reference. For brevity, not all features are expressly described and illustrated as combined with all other features.

### BRIEF DESCRIPTION OF THE DRAWINGS

The previous summary and the following detailed descriptions are to be read in view of the drawings, which illustrate some, but not all, embodiments and features as briefly described below. The summary and detailed descriptions, however, are not limited to only those embodiments and features explicitly illustrated.

FIG. 1 is a biologic scaffold tube coupled to a washer by a suture according to at least one embodiment.

FIG. 2 shows a needle withdrawing bone marrow aspirate.

FIG. 3 shows a dermal allograft consisting of the tube of FIG. 1 with concentrated bone marrow aspirate embedded into the interior side.

FIG. 4 shows a suture weaved through an anterior cruciate ligament in a locking fashion.

FIG. 5 shows a device passed through the medial portal of the knee in order to pull the ligament suture through the medial portal.

FIG. 6 shows the ligament suture pulled through the medial portal.

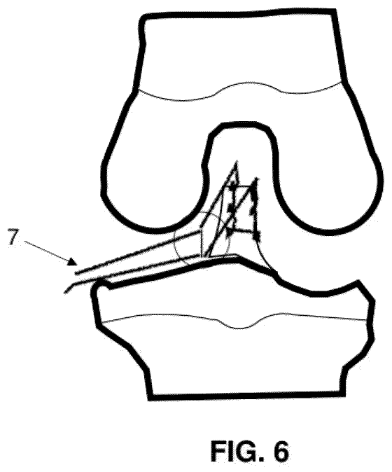

FIG. 7 shows a guide placed over the medial tibia in order to drill a hole.

FIG. 8 shows a guide placed in order to drill a hole.

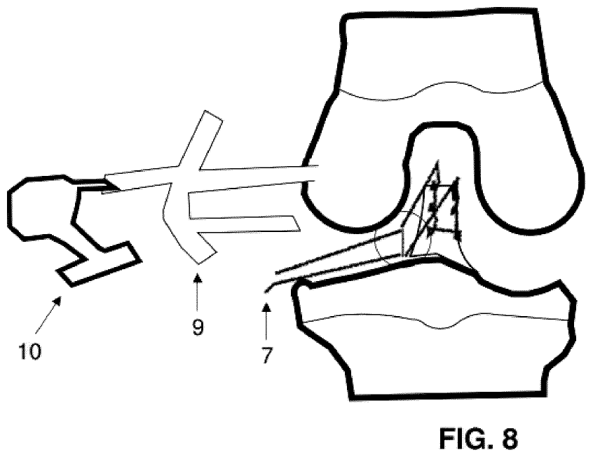

FIG. 9 shows a suture passed through the tibia tunnel.

FIG. 10 shows the free ends of the dermal tube sutures passed through the loop end of the tibia passing suture.

FIG. 11 shows the dermal tube sutures pulled through the tibia tunnel.

FIG. 12 shows the device of FIG. 5 in use.

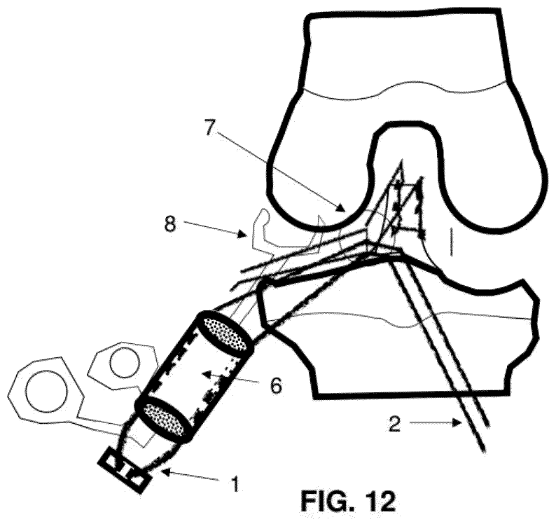

FIG. 13 shows the dermal allograft of FIG. 3 being inserted.

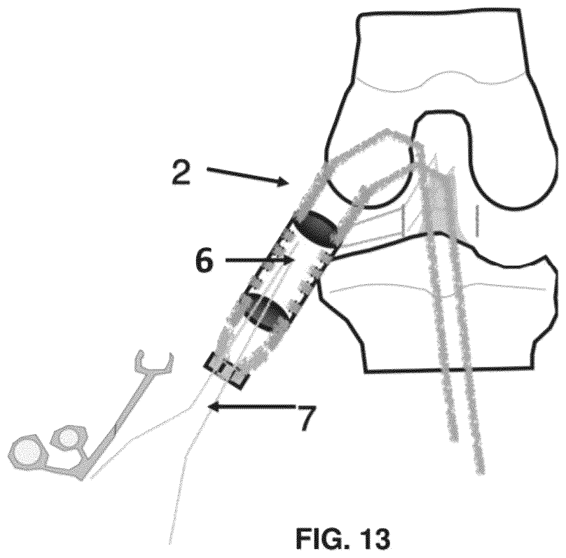

FIG. 14 shows a synthetic suture passed through the femur tunnel and the loop end pulled out through the medial portal.

FIG. 15 shows the ligament repair suture and washer suture.

FIG. 16 shows the washer suture passed through the femur tunnel.

FIG. 17 shows the washer inverted and placed on the tibia side with the device flipped upside down.

FIG. 18, shows an embodiment in which a knotless repair is done with two independent suture tapes running up both sides of the tube.

FIG. 19 shows an embodiment in which a knotless anchor is used instead of a button.

FIG. 20 shows a biologic scaffold sheet coupled to a washer by a suture according to at least one embodiment.

FIG. 21 shows the collagen interface in this embodiment as a rectangular sheet that runs next to the ACL when placed instead of around the ACL.

FIG. 22 shows the biologic scaffold sheet being placed.

### DETAILED DESCRIPTIONS

These descriptions are presented with sufficient details to provide an understanding of one or more particular embodiments of broader inventive subject matters. These descriptions expound upon and exemplify particular features of those particular embodiments without limiting the inventive subject matters to the explicitly described embodiments and features. Considerations in view of these descriptions will likely give rise to additional and similar embodiments and features without departing from the scope of the inventive subject matters. Although steps may be expressly described or implied relating to features of processes or methods, no implication is made of any particular order or sequence among such expressed or implied steps unless an order or sequence is explicitly stated.

Any dimensions expressed or implied in the drawings and these descriptions are provided for exemplary purposes. Thus, not all embodiments within the scope of the drawings and these descriptions are made according to such exemplary dimensions. The drawings are not made necessarily to scale. Thus, not all embodiments within the scope of the drawings and these descriptions are made according to the apparent scale of the drawings with regard to relative dimensions in the drawings. However, for each drawing, at least one embodiment is made according to the apparent relative scale of the drawing.

Any materials described are provided as non-limiting examples except where their inclusion is positively and unambiguously asserted. Elements described as metal can be, for example, made of surgical steel. Once materials and arrangements are described herein with reference to any structures and elements thereof, for example in the drawings, such descriptions apply as well to any further same or similar structures and elements that may appear in other drawings.

Like reference numbers used throughout the drawings depict like or similar elements. Unless described or implied as exclusive alternatives, features throughout the drawings and descriptions should be taken as cumulative, such that features expressly associated with some particular embodiments can be combined with other embodiments.

Unless defined otherwise, technical and scientific terms used herein have the same meaning as commonly understood to one of ordinary skill in the art to which the presently disclosed subject matter pertains.

The invention claimed here solves this problem.

A biologic tube reinforced with weaved suture for enhancing ligament or tendon repair with biologics and protecting repairs is disclosed.

This biologic tube provides a protected conduit for clotting and healing of the ligament to bone and additionally provides reinforcement of the repair to protect the repair site during healing. It also provides containment and placement of biologics including but not limited to concentrated bone marrow aspirate.

Also, it can produce It can be used to repair and reinforce repair of other ligaments or possibly tendons.

Additionally: It could be used to repair tissue in animals.

Reference numbers in the drawings refer to components and/or steps, each according to at least one embodiment, as in the below listing:
1 A metal washer with a small hole on the end for a passing suture and two slits in the middle for passing of a polyester or synthetic suture.
2 A thick polyester or synthetic suture or combination of bioabsorbable or biologic scaffold material with non-absorbable suture.
3 A biologic scaffold tube, collagen, dermal allograft tissue with hypodermal side towards the interior, or other biologic material.
4 A technique whereby the polyester or synthetic suture is weaved in and out of the sides of the tube through small perforations in the dermal allograft. The running suture weaves are 180 degrees from each other. A loop is made at one end. A modified technique can be used: Separately, a washer can be attached with a non-absorbable adjustable loop suture in a loop through the washer then through the loop of the tube suture 2 and back up through the washer.
5 A metal needle with impaction handle and syringe for withdrawing bone marrow aspirate.
6 A dermal allograft consisting of the tube 3 with concentrated bone marrow aspirate embedded into the interior side.
7 A polyester or synthetic suture weaved through the anterior cruciate ligament in a locking fashion.
8 A metal device used to grasp sutures and the ligament.
9 Metal guide which is adjustable to drill a hole in the tibia for passing the sutures through the tibia or femur.
10 A powered drill for making holes in the bone to pass suture.
11 A polyester or synthetic suture, passed through the tibia tunnel, used as a passing stitch for the tibia sided sutures.
12 A polyester or synthetic suture used to pass through the femur tunnel for pulling the suture attached to the ligament washer 1.
13 A polyester or synthetic suture attached to the washer 1, used to pass the button.
14 A metal washer with two holes in it to pass the suture 2 and tie it over, to secure it to the bone.
15 A flexible semi-rigid ring can be inserted into the tube at the tissue attachment side of the tube to keep the tube open. This can be made of a bioabsorbable material.

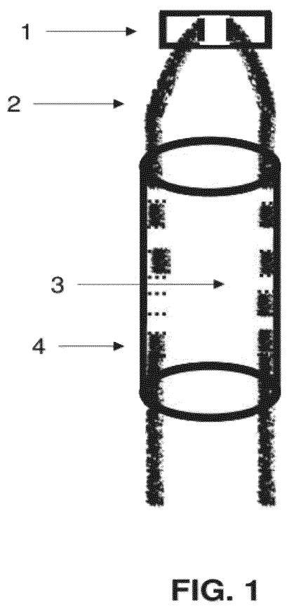

As shown in FIG. 1, a dermal allograft or collagen or biologic sheet is tubularized, with reference to the biologic scaffold tube 3. This is done, in at least one embodiment, by taking a rectangular allograft and rolling it into a tube, then suturing the edges together to secure the tube. This can be done to accommodate different size ligaments (i.e. 6, 7, 8, 9, 10, 11 mm diameter tubes). In an alternative embodiment, a biologic material can be created in the shape of a tube. A thick synthetic or synthetic/biologic suture 2 is passed up one side of the tube 3, weaving it in and out of a first side 4 of the tube 3. Then the suture 2 is passed through a metal washer 1 and passed back down the tube 3 in a similar fashion, weaving in and out of an opposite side.

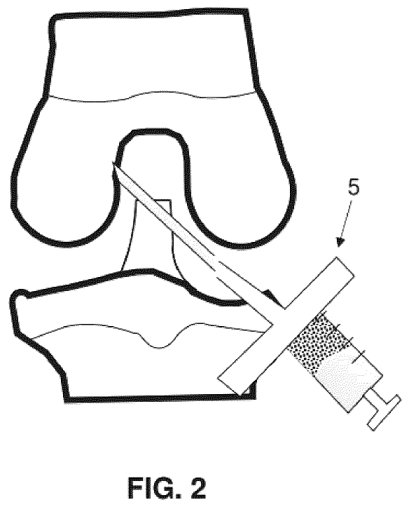

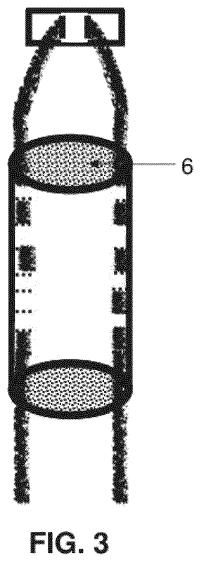

As shown in FIG. 2, a metal needle or trochar with attached impaction handle and syringe 5 is impacted into the femur and bone marrow aspirate is withdrawn from the femur. The withdrawn aspirate centrifuged to obtain concentrated bone marrow aspirate. The concentrated bone marrow aspirate is soaked over the graft to allow healing cells and factors to saturate the tube 3, producing the dermal allograft 6 (FIG. 3) with concentrated bone marrow aspirate.

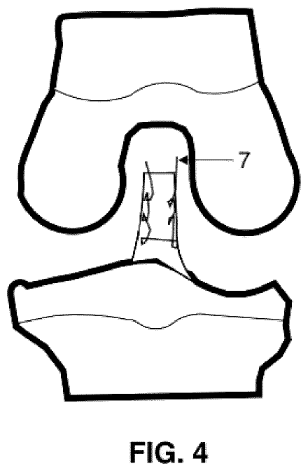

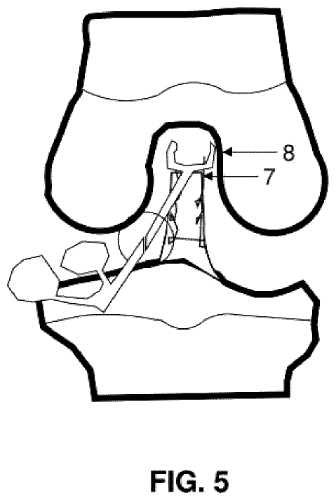

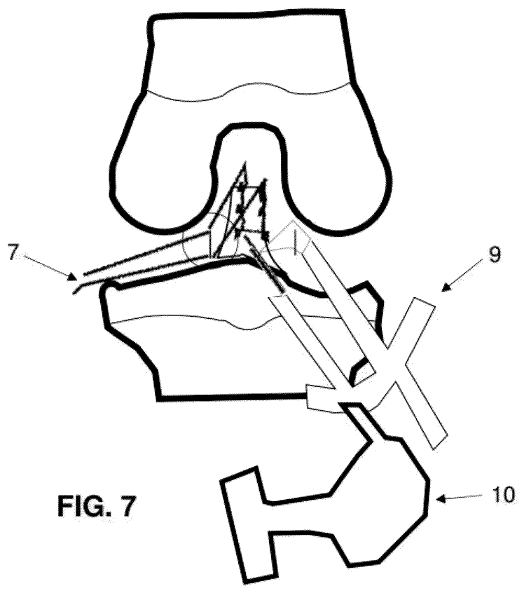

A synthetic suture 7 (FIG. 4) is weaved down and up the ligament to provide a stable suture in order to repair the ligament. A metal device 8 (FIG. 5) is passed through the medial portal of the knee in order to pull the ligament suture 7 through the medial portal. In FIG. 7, a metal guide 9 is placed over the medial tibia in order to drill a hole with a power drill 10 from the anteriormedial tibia into the tibia footprint of the anterior cruciate ligament or other ligament footprint. A cannulated reamer is used over the pin to enlarge the hole if needed.

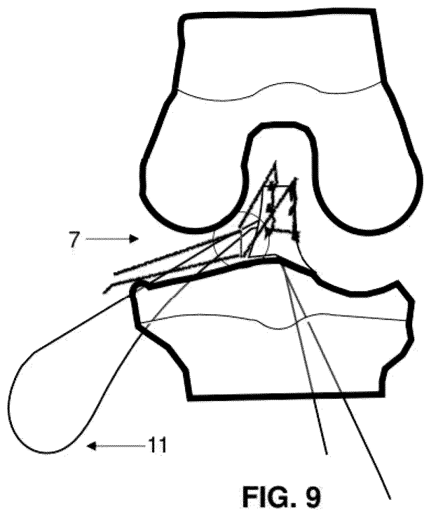

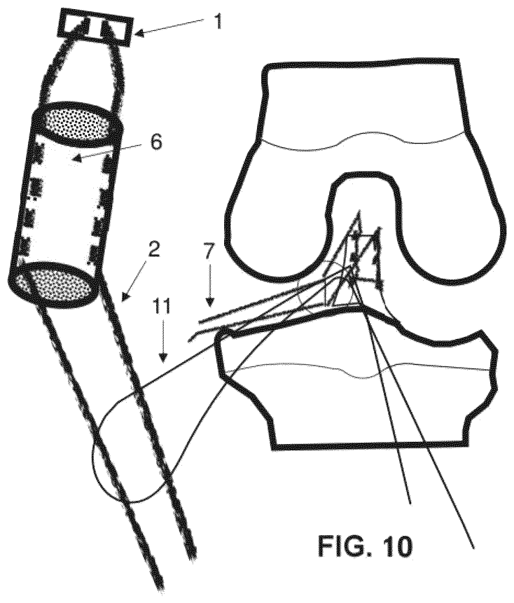

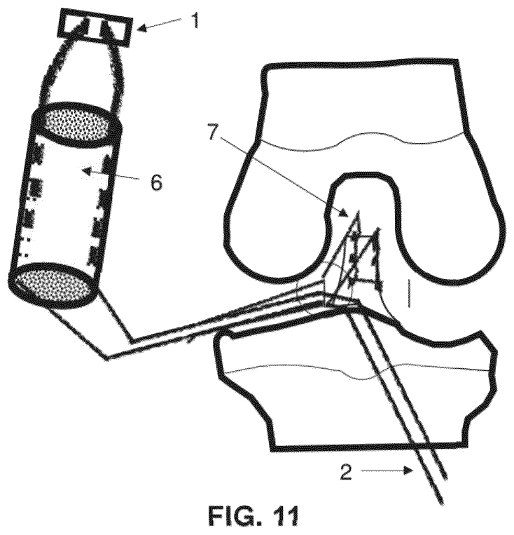

A synthetic passing suture 11 (FIG. 9) is passed through the tibia tunnel. The loop end (FIG. 10) of the tibia passing suture 11 is pulled through the medial knee portal and used to loop and pull the free ends of the dermal tube sutures 2. The dermal tube sutures 2 are pulled through the tibia tunnel in FIG. 11.

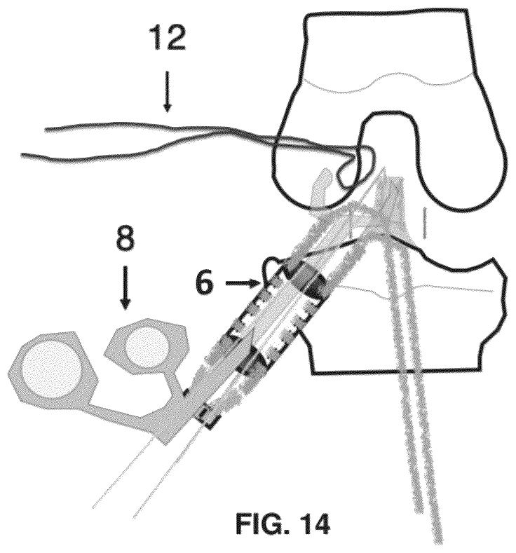

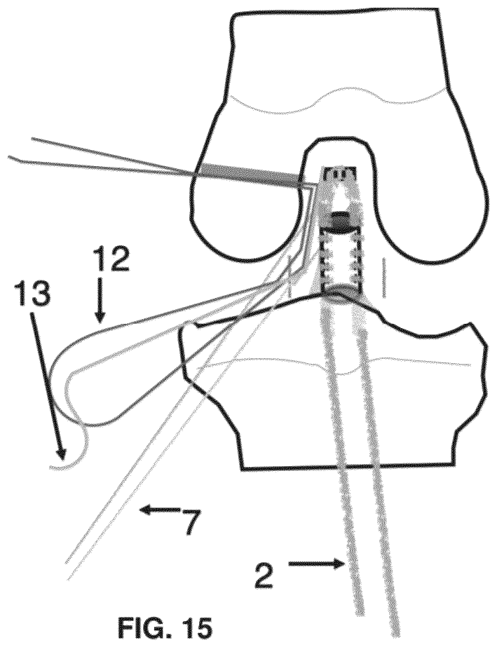

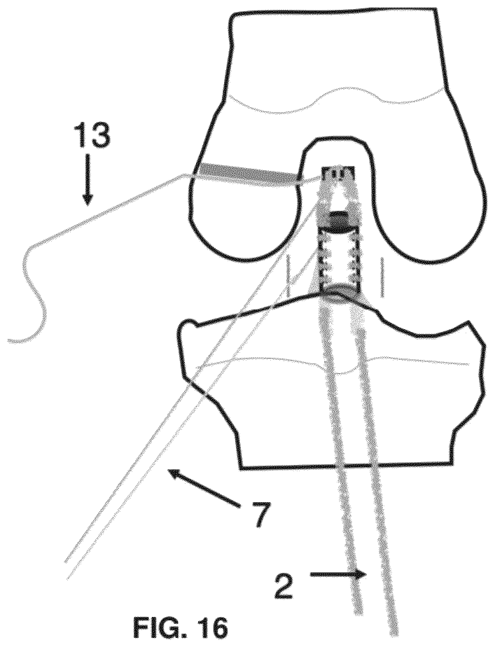

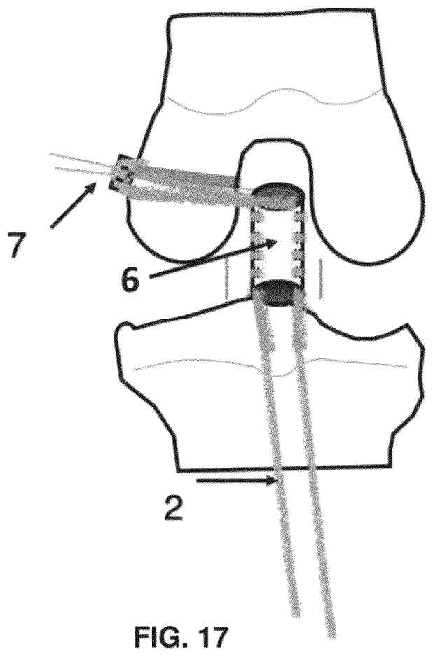

A synthetic suture 12 (FIG. 14) is passed through the femur tunnel and the loop end is pulled out through the medial portal. The ligament repair suture 7 and washer suture 13 are pulled through the medial portal (FIG. 15). The femur passing suture 12 is used to pass the ligament repair suture 7 and washer suture 13 through the femur tunnel (FIG. 16). Once the washer is pulled through the femur tunnel and rotated 90 degrees and it is stable, the ligament repair suture 7 is secured to the passing suture 13. The free ends of the tube sutures 2 are then secured and tied over a metal washer on the tibia side. The metal washer 1 can be inverted and placed on the tibia side with the device flipped upside down (FIG. 17).

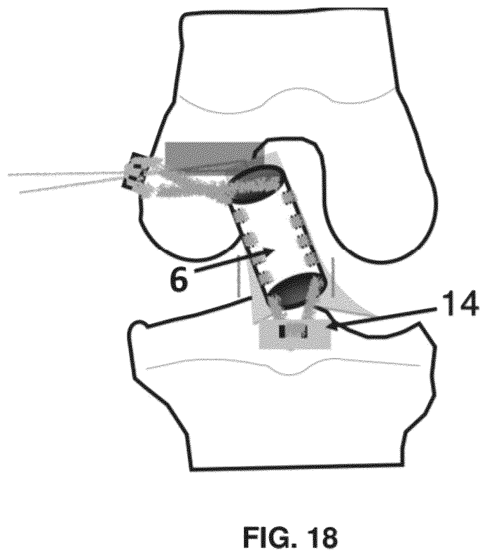

Additionally, as shown in FIG. 18, a knotless repair can be done with two independent suture tapes running up both sides of the tube. Each suture can be repaired to bone independently to attach the ends of the tube at two separate points on bone.

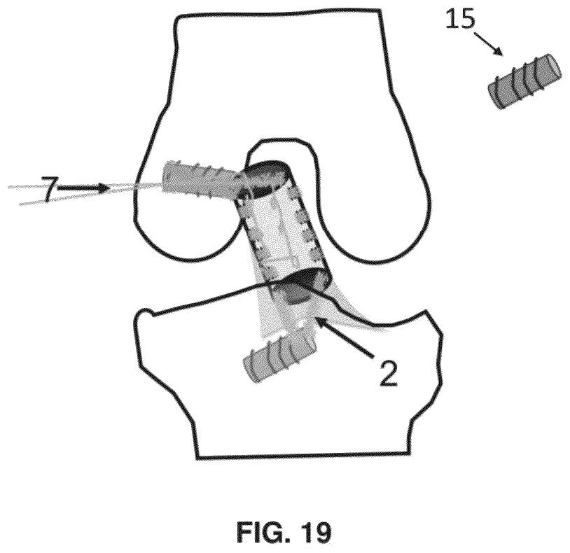

FIG. 19 shows an embodiment in which a knotless anchor is used instead of a button. Knotless screw fixation can be used instead of button. A knotless screw or knotless all suture anchor 15 can be used to affix the sutures attached to the ligament/tendon 7 and the suture attached to the sleeve 2.

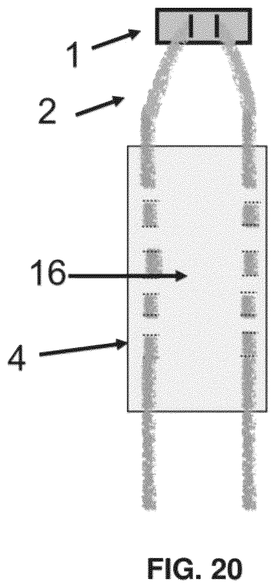

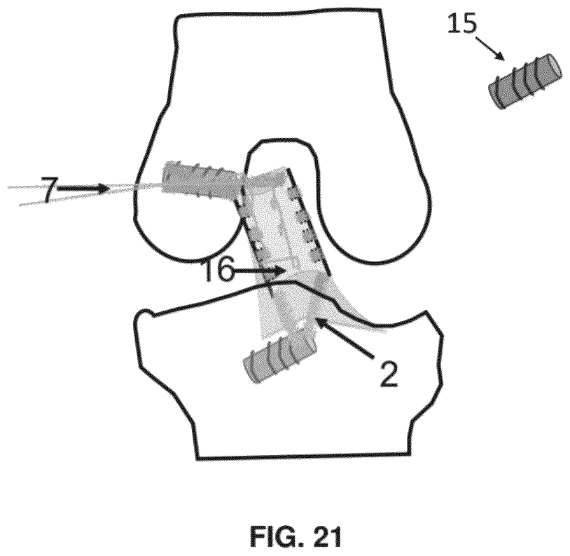

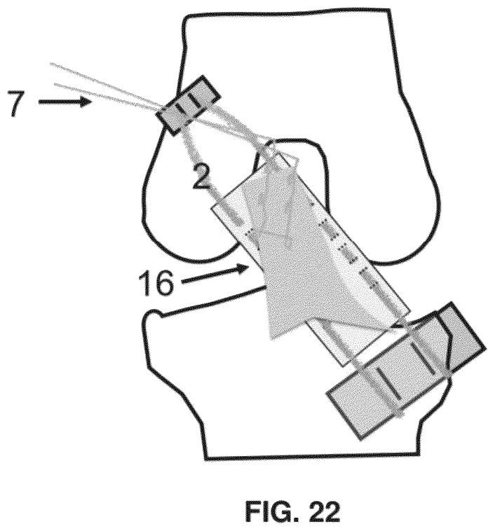

FIGS. 20-22 illustrate an embodiment in which a sheet is used instead of a tube or sleeve. FIG. 20 shows a biologic scaffold sheet 16 is coupled to a washer by a suture according to at least one embodiment. FIG. 21 shows the collagen interface in this embodiment as a rectangular sheet 16 that runs next to the ACL when placed instead of around the ACL. FIG. 22 shows the biologic scaffold sheet 16 being placed. The sheet 16 and sutures 2 and 7 can be affixed with buttons.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

What is claimed is:

1. A medical device comprising:
a scaffold having at least two sides;
a washer and/or knotless anchor; and
at least one suture or suture tape having a medial portion passed through the washer and/or affixed to the knotless anchor, and two portions extending from the medial portion each weaved to a respective one of the two sides of the scaffold.

2. The medical device of claim 1, wherein the scaffold comprises at least one of collagen and/or dermal allograft tissue.

3. The medical device of claim 1, wherein the scaffold comprises a tube.

4. The medical device of claim 3, wherein bone marrow aspirate is embedded into an interior side of the tube.

5. The medical device of claim 3, wherein two portions extending from the medial portion are weaved to respective two diametrically opposed sides of the tube.

6. The medical device of claim 3, further comprising a ring inserted into the tube at a tissue attachment side of the tube to keep the tube open.

7. The medical device of claim 3, wherein the ring comprises a bioabsorbable material.

8. The medical device of claim 1, wherein the scaffold comprises a rectangular sheet or two separate rectangular sheets with one suture weaved through each separate sheet.

9. The medical device of claim 1, wherein the scaffold includes bone marrow aspirate.

10. The medical device of claim 1, wherein the suture or suture tape is passed through the washer; and
the washer has a hole on an end thereof for a passing the at least one suture or suture tape and two slits in a middle thereof.

11. A medical procedure comprising repairing a ligament or tendon using the medical device of claim 1.

12. A medical procedure comprising placing the tube of claim 3 around an ligament or tendon.

13. A medical procedure comprising placing the rectangular sheet of claim 8 next to an ligament or tendon.
